(19) United States
(12) Patent Application Publication
(10) Pub. No.: US 2024/0058268 A1
(43) Pub. Date: Feb. 22, 2024

(54) METHODS OF TREATING SKIN DISORDERS

(71) Applicant: GALDERMA HOLDING SA, Zug (CH)

(72) Inventors: Valerie JULIA, Biot (FR); Jayendra Kumar KRISHNASWAMY, Pully (CH)

(73) Assignee: GALDERMA HOLDING SA, Zug (CH)

(21) Appl. No.: 18/237,864

(22) Filed: Aug. 24, 2023

Related U.S. Application Data
(62) Division of application No. 17/666,252, filed on Feb. 7, 2022.
(60) Provisional application No. 63/146,960, filed on Feb. 8, 2021.

(51) Int. Cl. A61K 9/107; A61P 17/10; A61K 31/40

(52) U.S. Cl. CPC A61K 9/107; A61P 17/10; A61K 31/40

(57) ABSTRACT

Disclosed herein are methods for treatment of a skin disorders using pharmaceutical compositions that reduces the expression of and/or inhibit the activity of various genes including chemokine (C-X-C motif) ligands such as CXCL13 and XCL1, and matrix-metalloproteinases such as MMP12 and MMP13. The pharmaceutical compositions effectively act on epidermal differentiation and proliferation as well as on innate and adaptive immune cells in pathogenesis of skin disorders such as acne.

### CROSS-REFERENCE TO RELATED APPLICATIONS

This application is a divisional application of U.S. patent application Ser. No. 17/666,252, filed Feb. 7, 2022, which application claims priority to and benefit of U.S. Provisional Patent Application No. 63/146,960 filed on Feb. 8, 2021, the content of both is incorporated herein by reference in its entirety.

### FIELD

Described herein are methods for treating or alleviating symptoms associated with a skin disorder such as acne vulgaris, using pharmaceutical compositions and agents which inhibit or reduce the expression one or more genes such as CXCL13, XCL1, SPP1, MMP12 and MMP13.

### BACKGROUND

The following discussion is provided to aid the reader in understanding the disclosure and is not admitted to describe or constitute prior art thereto.

Acne vulgaris (AV) is one of the most common skin disorder, and it has a multifactorial, complex pathogenesis that centers on pilosebaceous units. In acne-prone skin, inflammatory processes may precede the development of a visibly apparent lesion in skin that appears otherwise clinically normal. Thus, targeting inflammation is an essential part of acne therapy.

Compounds with activity of retinoid type (vitamin A and its derivatives) are widely known for their potential in the treatment or prevention of dermatologic conditions, including acne. Several of the biological effects of retinoids are mediated by modulating the nuclear retinoic acid receptors (RAR), which activate transcription by binding to DNA sequence elements, known as RAR response elements (RARE), in the form of a heterodimer with the retinoid X receptors (known as RXRs). Three subtypes of human RARs have been identified and described: RARα, RARβ and RARγ. Conventional topical retinoids have differing receptor binding profiles to retinoic acid receptor (RAR) subtypes, which may translate to clinical differences and likely affect suites of genes that could result in class effects (such as normalization of epithelial turnover) as well as specific functional responses to an individual retinoid, particularly in activation or suppression of inflammatory pathways.

There remains a need to develop novel therapeutic regimes to treat patients with skin disorders, particularly those suffering from acne, including acne vulgaris taking in to consideration the genetic processes and regulatory mechanisms at the molecular level and how variations in gene expression and receptor binding may translate to differences in the clinical treatment.

### SUMMARY

Provided herein are methods for treating a skin disorder in a subject and pharmaceutical compositions for use in the treatment of a skin disorder, such as acne vulgaris.

In accordance with one or more embodiments, there are provided methods for treatment of a skin disorder in a subject, the method comprising administering to said subject a pharmaceutical composition that inhibits CXCL13 activity.

In accordance with one or more embodiments, there are provided methods for treatment of a skin disorder in a subject, the method comprising administering to said subject a pharmaceutical composition that inhibits MMP12 and/or MMP13 activity.

In accordance with one or more embodiments, there are provided methods for treatment of a skin disorder in a subject, the method comprising administering to said subject a pharmaceutical composition exhibiting combined CXCL13 inhibitory activity and MMP12 and/or MMP13 inhibitory activity.

In one or more embodiments, the pharmaceutical composition comprises a therapeutically effective amount of a pharmaceutical agent and a pharmaceutically acceptable carrier. In one or more embodiments, the pharmaceutical agent inhibits the activity of or reduces the expression of one or more genes selected from CXCL13, MMP12 and MMP13.

In accordance with one or more embodiments, there are provided methods for treatment of a skin disorder in a subject in need thereof, the method comprising administering to a subject in need thereof, a pharmaceutical composition, wherein said pharmaceutical composition reduces the expression of one or more genes selected from CXCL13, MMP12 and MMP13.

In accordance with one or more embodiments, there are provided methods for treatment of a skin disorder in a subject, the method comprising administering to said subject a pharmaceutical composition that reduces the expression of CXCL13.

In accordance with one or more embodiments, there are provided methods for treatment of a skin disorder in a subject, the method comprising administering to said subject a pharmaceutical composition that reduces the expression of MMP12 and/or MMP13.

In accordance with one or more embodiments, there are provided methods for treatment of a skin disorder in a subject, the method comprising administering to said subject a pharmaceutical composition that reduces the expression of combined CXCL13 and MMP12 and/or MMP13.

In one or more embodiments, the pharmaceutical composition comprises a therapeutically effective amount of a pharmaceutical agent and a pharmaceutically acceptable carrier. In one or more embodiments, the pharmaceutical agent reduces the expression of one or more genes selected from CXCL13, XCL1, SPP1, MMP12 and MMP13.

In accordance with one or more embodiments, there are provided methods for treatment of a skin disorder in a subject in need thereof, the method comprising administering to a subject in need thereof, a pharmaceutical composition, wherein said pharmaceutical composition reduces the expression of one or more genes selected from CXCL13, XCL1, SPP1, MMP12 and MMP13.

In one or more embodiments, the pharmaceutical composition comprises from about 1 μg/g to about 100 μg/g of a pharmaceutical agent. In one or more embodiments, the pharmaceutical agent is administered once daily, twice daily, once per week, once every two weeks, once every three weeks, once every four weeks, once every five weeks, once every six weeks, once every seven weeks, or once every eight weeks. In one or more embodiments, the pharmaceutical composition is formulated as a cream or as an oil-in-water emulsion. In one or more embodiments, the pharmaceutical composition is formulated as a cream.

In one or more embodiments, the subject has acne. In one or more embodiments of the methods, the skin disorder is acne vulgaris. In one or more embodiments of the methods, the skin disorder is acne vulgaris of the face or trunk. In one or more embodiments of the methods, the acne vulgaris comprises mild, moderate and severe acne vulgaris. In one or more embodiments, the pharmaceutical composition is administered topically to the area of the subject affected with acne vulgaris.

In one or more embodiments, there are provided methods of modulating an adaptive immune response in a subject to effect a treatment of a skin disorder, comprising administering to the subject a pharmaceutical composition that inhibits the activity of or reduces the expression of one or more genes selected from CXCL13, XCL1, SPP1, MMP12 and MMP13.

In one or more embodiments, there are provided methods of alleviating inflammatory response associated with skin disorders in a subject, comprising administering to the subject a pharmaceutical composition that inhibits the activity of or reduces the expression of one or more genes selected from CXCL13, XCL1, SPP1, MMP12 and MMP13. In one or more embodiments, the skin disorder is acne vulgaris and the method comprises treating acne vulgaris. In one or more embodiments, the method decreases inflammation in the skin of the subject.

In one or more embodiments, there are provided methods of antagonizing CXCR5 receptor activity in a patient suffering from acne vulgaris, comprising administering to the patient in need thereof a therapeutically effective amount of a pharmaceutical agent which inhibits the activity of or reduces the expression of CXCL13.

In one or more embodiments, there are provided methods of monitoring or determining efficacy of an acne treatment in a subject, the method comprising determining in a sample from said subject the expression of one or more genes selected from CXCL13, XCL1, SPP1, MMP12 and MMP13. In one or more embodiments, the subject is treated with a pharmaceutical composition that inhibits the activity of or reduces the expression of one or more genes selected from CXCL13, XCL1, SPP1, MMP12 and MMP13. In one or more embodiments of the method, a decrease or an increase in the expression of one or more genes selected from CXCL13, XCL1, SPP1, MMP12 and MMP13 in the sample relative to the controls is used to determine the efficacy of the acne treatment.

### BRIEF DESCRIPTION OF THE DRAWINGS

FIG. 1 illustrates methodology for biopsy sampling in acne patients.

FIG. 2 illustrates a Venn diagram comparing the number of candidates for differentially expression genes following the analyses of trifarotene treatment vs spontaneously resolved papule signature (NPS-R3 vs NPS-L1), the papule signature (NPS-L1 vs NPS-LNI), and the signature of a papule after vehicular treatment (NPS-L3 vs NPS-L1).

FIG. 3 illustrates genes which are uniquely downregulated by trifarotene and involved in inflammatory cell infiltration and extracellular matrix reorganization

FIG. 4 illustrates the ingenuity pathway analysis of trifarotene specific gene signature.

FIGS. 5 (A) and 5 (B) illustrates open target analyses of trifarotene and papule signature showing the diseases and pathways regulated by trifarotene specific genes.

FIG. 6 (A) illustrates single cell data analysis of normal and acne skin biospies (GSE150672). UMAP representation of the dataset showing the main cell types detected, and FIG. 6 (B) illustrates changes in expression levels of marker genes associated with different cell populations including Fibroblasts, melanocytes, venular cells, B cells, mast cells and myeloid cells.

FIG. 7 illustrates cell-type-specific gene signatures distribution, wherein (A) Overview of changes in expression for major cell types present in skin biopsies, and (B-E) illustrates changes in expression levels of marker genes associated with different classes of macrophages.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

Unless the context indicates otherwise, it is specifically intended that the various features of the invention described herein can be used in any combination. Moreover, the disclosure also contemplates that in one or more embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

### Definitions

As used herein, the singular forms “a,” “an,” and “the” designate both the singular and the plural, unless expressly stated to designate the singular only.

It is to be understood, although not always explicitly stated, that all numerical designations are preceded by the term “about.” The term “about” means that the number comprehended is not limited to the exact number set forth herein, and is intended to refer to numbers substantially around the recited number while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 15%, 10%, 5%, 1%, or 0.1% of the particular term.

Also as used herein, “and/or” refers to and encompasses any and all possible combinations of one or more of the associated listed items, as well as the lack of combinations when interpreted in the alternative (“or”). Thus, acne vulgaris includes acne vulgaris of the face, acne vulgaris of the trunk, or acne vulgaris of the face and trunk.

The terms “administer,” “administration,” or “administering” as used herein refer to (1) providing, giving, dosing and/or prescribing, such as by either a health professional or his or her authorized agent or under his direction, and (2) putting into, applying, taking or consuming, such as by a health professional or the subject. For example, administration can include without limitation, topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

The terms “treat”, “treating” or “treatment”, as used herein, include alleviating, attenuating, abating or ameliorating skin disorders, or one or more symptoms thereof, whether or not the skin disorder is considered to be “cured” or “healed” and whether or not all symptoms are resolved. The terms also include reducing or preventing progression of a skin disorder or one or more symptoms thereof, impeding or preventing an underlying mechanism of the skin disorder or one or more symptoms thereof, and achieving any therapeutic and/or prophylactic benefit.

As used herein, the term “subject” is used interchangeably with “patient,” and indicates a mammal, in particular a human, equine, bovine, porcine, feline, canine, murine, rat, or non-human primate. In one or more embodiments, the subject is a human.

As used herein, the term “equivalent thereof” as used herein, include, for example, salts, precursors, derivatives, esters, polymorphs, etc. of the therapeutic active agent. For example, an equivalent of trifarotene can include pharmaceutically acceptable salts thereof.

The term “precursors” means the immediate biological precursors or substrates thereof, and also the chemical precursors thereof.

The term “derivatives” means both the metabolic derivatives thereof and the chemical derivatives thereof.

An “effective amount” is an amount sufficient to effect beneficial or desired results such as alleviating at least one or more symptom of a skin condition or disorder, such as for example, acne vulgaris. An effective amount as used herein would also include an amount sufficient to delay the development of or alter the course of the skin disorder and/or a symptom (for example low self-esteem), or reverse a symptom of the skin disorder. Thus, it is not possible to specify the exact “effective amount.” However, for any given case, an appropriate “effective amount” can be determined by one of ordinary skill in the art using only routine experimentation. For example, the effective amount of pharmaceutical agent or composition may include the use of a thin layer of a topical composition sufficient to cover the area to be treated.

In one or more embodiments, the skin disorder such as acne vulgaris are scored as clear, almost clear, mild, moderate, or severe. “clear,” “almost clear,” “mild,” “moderate,” and “severe” are terms of art in describing the presence, extent, anatomical location, extent on the body, type of morphological lesions (e.g., papules, pustules, nodules, cysts, scars, comedones, etc.), severity, and/or intensity of the acne. Those of skill in the art know the metes and bounds of these terms.

The term “carrier” refers to a diluent, adjuvant, excipient, or vehicle with which the therapeutic agent is administered.

Inflammatory skin disorders are well-known and may include acne, psoriasis, hives, eczema, rosacea, vitiligo, keratosis and dermatitis. Acne vulgaris is a common chronic inflammatory skin disease with a complex pathogenesis developing in the sebaceous follicles. Four key pathogenic factors have been identified: (i) an alteration in the pattern of keratinisation within the follicle, (ii) increased sebum production, (iii) colonization with Propionibacterium acnes, and (iv) inflammation. In acne-prone skin, inflammatory processes may precede the development of a visibly apparent lesion in skin that appears otherwise clinically normal; thus, targeting inflammation is an essential part of acne therapy. Topical retinoids interact with nuclear receptors to affect gene transcription; specifically, their actions are mediated by retinoic acid receptors (RARs) and retinoid X receptors (RXRs). Retinoic acid (RA) or a synthetic RAR agonist, (e.g., trifarotene) binds to RAR/RXR, enabling the formation of a co-activator complex and subsequent changes in the expression of downstream genes. The anti-inflammatory effects of topical retinoids have been well established, particularly in regards to the innate immune response. The present technology provides methods by which reducing the expression of certain genes, exhibits a direct effect on the adaptive immune system and helps attenuate the inflammatory response in skin disorders such as acne.

The following abbreviations are used herein: LS=Lesional Skin, L1=LS Before Treatment, L3=LS After Vehicle Treatment, R3=LS After CD5789 Treatment, L-NI=Non Involved Skin, PS=Prone Scare, and NPS=Non Prone Scare.

### Therapeutic Active Agent

The therapeutic active agent or pharmaceutical agent to be used according to the technology include compounds which reduce the expression of one or more genes associated with reversal of inflammatory pathways or matrix reorganization. Examples of such genes include, but are not limited to chemokine (C-X-C motif) ligands such as e.g., CXCL13, secreted phosphoproteins such as e.g., SPP1, and members of the matrix-metalloproteinase family such as e.g., MMP12 and MMP13. Other examples of suitable genes are summarized in Table 1. Suitable compounds which can reduce the expression of more genes associated with reversal of inflammatory pathways or matrix reorganization, which optionally have inhibitory activity against such genes and which can be used in the treatment of acne include, for example, retinoids, including compounds which are retinoid acid or a synthetic retinoic acid receptor (RAR) agonists. One such example of a retinoid acid receptor γ (RARγ) agonist is trifarotene, which is a terphenyl acid derivative having the chemical name 3″-tert-Butyl-4′-(2-hydroxy-ethoxy)-4″-pyrrolidin-1-yl-[1,1′,3′,1″ ]terphenyl-4-carboxylic acid. Trifarotene is marketed under the trademark AKLIEF® at a concentration of 50 micrograms per gram (mcg/g or μg/g), in the form of a cream. In an embodiment, the therapeutic active agent or pharmaceutical agent is trifarotene. In an embodiment, the therapeutic active agent or pharmaceutical agent is not trifarotene.

The present inventors unexpectedly discovered that in addition to promoting epidermal differentiation and keratinization processes, retinoid acid receptors uniquely downregulate genes involved in inflammatory cell infiltration (e.g., CXCL13, XCL1) and extracellular matrix reorganization (e.g., SPP1/Osteopontin, MMP12, MMP13). It was also discovered that the LXR/RXR signalling is downregulated. These heretofore-unknown activities were evaluated using trifarotene as an exemplary pharmaceutical agent and acne vulgaris as an exemplary skin condition.

Subjects with moderate inflammatory acne of the back were treated with a 0.005% trifarotene or vehicle cream on dedicated back areas for 27 days, and 4 biopsies were collected on each subject (non-involved skin, acne papule, Trifarotene and vehicle treated site). Large scale gene expression profiling of the biopsies was performed using Affymetrix technology, and treatment specific gene expression profiles were generated using statistical modeling.

### (I) Transcriptome Data and Relevance of Bioinformatic Analysis

To gain an enhanced understanding of how trifarotene exerts its effects in acne, a post-hoc bioinformatics analysis was performed on transcriptomics data collected from lesional and non-lesional skin of patients with acne vulgaris with the goal of identifying genes or pathways, using the action of trifarotene as an example. The study is pioneering and compares gene expression in normal skin, spontaneously resolving acne lesions, and topical-retinoid-treated acne lesions. Bioinformatic analyses are performed to extract useful information from large amounts of raw data. Biologic and genetic data stored in sequence databases can be organized and queried. This allows study of how normal cellular activities change in individual disease states.

### (II) Clinical Open-Label Study

Clinical investigations were conducted in accordance with the Declaration of Helsinki principles, and the ICH Guideline for Good Clinical Practice. The clinical study received approval from the ethics committee of Brest, France (reference CPP Quest 6-755). This was a post-hoc analysis of data collected from 9 patients aged 18-35 with acne vulgaris (EUDRACT No. 2012-001943-36). In the 4-week open-label clinical study, subjects received once-daily applications of trifarotene 0.005% cream and vehicle cream on the back. Each patient had four biopsies performed, 1 from skin without visible acne lesion (non-involved skin) and three at the site of acne papule, with the following timing: one prior to treatment (day 0), one after vehicle treatment (day 27), and one after trifarotene treatment (day 27). Aubert et al, Br J Dermatol., 2018 August; 179(2):442-456.

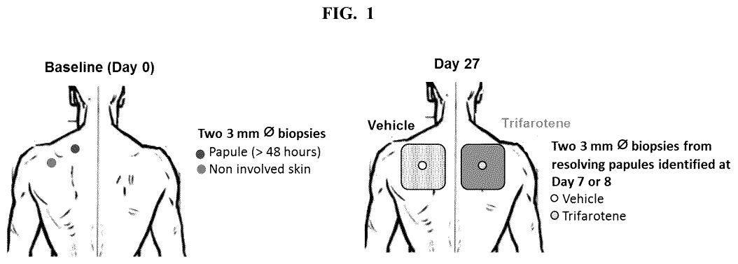

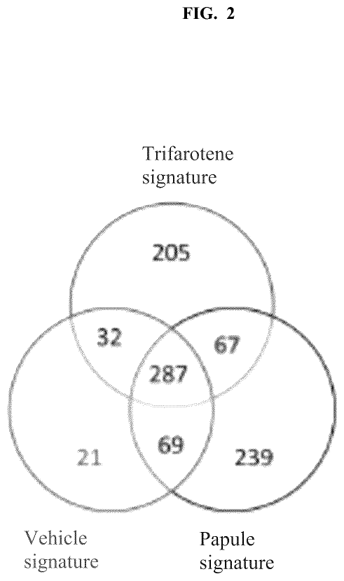

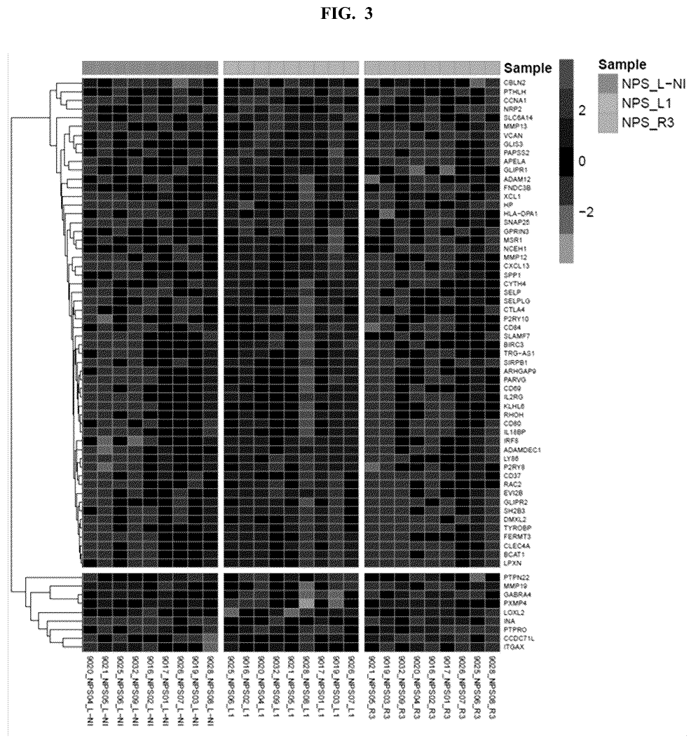

Samples were collected for large-scale gene expression profiling. Subjects provided written informed consent prior to biopsies. Subjects with moderate inflammatory acne on the back at the screening visit, defined by scores of between 2 and 4 for the whole back, with at least one area scored at 2 and a maximum of three nodules using the ECLA (Acne Lesion Score Scale) scale, were included in the study. Each subject had at least 2 papules 48 hours old, one on each half of the back. Subjects were excluded if they had active skin disease or inflammation other than acne, underlying known conditions which could interfere with study results, history of allergy to local anesthetics and/or topical antiseptics, history of bleeding disorder, or pregnancy/lactation (females). Predefined washout periods were mandated for acne treatments, immunosuppressants, immunomodulators, systemic corticosteroids, and isotretinoin. Study personnel applied treatment to patients' backs with trifarotene cream (right side) or vehicle (left side) once daily for 28 days excluding weekends (n=19 applications). Biopsies of nonlesional areas of trifarotene- and vehicle-treated skin were made under local anaesthesia at day 27 as shown in FIG. 1. Biopsies were stored in RNAlater TissueProtect Tubes (Qiagen, Les Ulis, France).

### (III) Analysis

Bioinformatics Analysis: Data analysis was performed using Array Studio software (Omicsoft Corporation, Cary, NC, USA). Affymetrix U133 Plus 2.0 chips (GeneChip™, Santa Clara, CA, USA) were normalized using the multi-array average (RMA) method (See e.g., Irizarry R A, et al. Exploration, normalization, and summaries of high density oligonucleotide array probe level data. Biostatistics, 2003; 4:249-64.) Low expression probe-sets were filtered. Only Affymetrix identifiers with expression ≥2⁶ in at least 5 samples in one condition were selected for further statistical analysis, resulting in data for 36245 out of the 54675 probe-sets present on the HG U133 Plus 2.0 array. Mean expression levels were obtained by calculating the geometric means of the RMA-normalized data. A linear model was fitted to the normalized data, and statistical testing of the comparisons of interest were performed using a moderated t-test. Paired-test was used when applicable. P values were adjusted for multiple testing using the Benjamini-Hochberg false discovery rate (FDR) method (See e.g., Benjamini Y, et al., Controlling the false discovery rate in behavior genetics research. Behav Brain Res, 2001; 125:279-84). Candidate differentially expressed genes were selected using a combination of fold-change and FDA; genes with absolute fold change>2 and FDA<0.05 were considered as candidate differentially expressed genes.

Functional and network analysis were generated with the Ingenuity Pathway Analysis software (QIAGEN® Inc., qiagenbioinformatics.com/products/ingenuity-pathway-analysis), and with Open Targets (opentargets.org/). IPA was used to identify canonical pathways, diseases and functions, and gene networks related to differentially expressed genes in acne lesions and post-lesional skin after treatment with trifarotene or vehicle. In addition, gene set enrichment analysis was performed with the R package FGSEA (version 1.12.0) using the hallmark and reactome gene sets.

Single-Cell Analysis and Cell Marker Definition: Single-cell RNAseq data from Hughes et al. was used to determine the major cell types present in skin biopsies and the associated cell markers. Acne and normal samples from GSE150672 were downloaded and analyzed using the Seurat package. Data were normalized, scaled, and log-transformed. The most variable genes were selected based on their expression and dispersion. To reduce dimensionality, principal component analysis (PCA) was applied. The first eight principal components were used to cluster the cells using the graph-based approach FindClusters function in Seurat, with a resolution parameter of 0.4. Graphical representation of cell clusters was achieved using UMAP. A combination of known markers and differentially expressed genes was used to characterize cell clusters. Major cell type markers retained for scoring analysis included: B cells: MS4A1, CD79A; Fibroblasts: DCN, COL6A2, APOD, CFD, IGFBP5, COL1A2, COL1A1, COL3A1; Keratinocytes: KRT5, KRT1, KRT14, KRT15, S100A2, KRT6A, HOPX, KRT10, DSP; Langerhans cells: CD207; Mast cells: CPA3, IL1RL1, CTSG, TPSAB1, GATA2; Melanocytes: MLANA, MITF, PMEL, DCT; Myeloid cells: CD68, CTSS; Schwann cells: SCN7A; T cells: CD3D, TRBC2, IL7R, PTPRC, CXCR4; Venular cells: SELE, CD93, TM4SF1, A2M, RCAN1; Vascular smooth muscle cells (VSMC): TAGLN, RGS5, MYH11, ACTA2, MYL9.

Macrophage Gene Signatures: Gene markers specific to each class of macrophage were derived from the literature. For M0, M1 and M2 macrophages, the markers described in Newman et al. (Robust enumeration of cell subsets from tissue expression profiles, Nat Methods, 12:453-7, 2015) were used. For SPP1 activated macrophages, the markers from Morse et al. (Proliferating SPP1/MERTK-expressing macrophages in idiopathic pulmonary fibrosis. Eur Respir J., 54:1802441) were used. Final markers were as listed. M0 macrophage: ACP5, BHLHE41, C5AR1, CCDC102B, CCL22, CCL7, COL8A2, CSF1, CXCL3, CXCL5, CYP27A1, DCSTAMP, GPC4, HK3, IGSF6, MARCO, MMP9, NCF2, PLA2G7, PPBP, QPCT, SLAMF8, SLC12A8, TNFSF14, VNN1. M1 macrophage: ACHE, APOBEC3A, APOL3, APOL6, ARRB1, CCL19, CCL5, CCR7, CD38, CD40, CHI3L1, CXCL10, CXCL11, CXCL13, CXCL9, CYP27B1, DHX58, HESX1, IDO1, IFI44L, IL2RA, KIAA0754, KYNU, LAG3, LAMP3, LILRA3, LILRB2, NOD2, PLA1A, PTGIR, RASSF4, RSAD2, SLAMFI, SLC2A6, SOCS1, TLR7, TNFAIP6, TNIP3, TRPM4. M2 macrophage: AIF1, ALOX15, CCL13, CCL14, CCL23, CD209, CD4, CFP, CLEC10A, CLEC4A, CRYBB1, FES, FRMD4A, FZD2, GSTT1, HRH1, HTR2B, MS4A6A, NME8, NPL, P2RY13, PDCD1LG2, RENBP, WNT5B. SPP1 macrophage: MERTK, CD14, SPP1, CD68, LYZ.

Cell Type Scoring: Each cell type gene signature was used to define a cell type average gene expression in each of our clinical study samples, calculated as the average of the log 2 expression values of these markers. Boxplots were used to visualize the distribution of cell type gene expression scores between clinical groups. Statistical significance was assessed using Wilcoxon rank sum test. Graphs were generated using the ggpubr data visualization library.

### (IV) Results

A comparative analysis was performed to identify expression patterns: For this analysis, the following comparisons were made to develop characteristic expression patterns: (a) Papule before treatment vs non-involved skin was used to derive an acne signature; (b) Resolved papule zone after treated with vehicle vs papule before treatment was used to derive signature of spontaneously resolved papule; and (c) Resolved papule zone after treatment with trifarotene vs papule before treatment to determine trifarotene treatment signature. To assess the mode of action of trifarotene, first gene expression profiles were derived of (1) acne lesion at baseline (papule signature), (2) previously lesional skin after spontaneous resolution of the papule (vehicle signature), and (3) previously lesional skin after resolution of a papule following trifarotene treatment (trifarotene signature). Papule signature was defined as genes differentially expressed when comparing the papule biopsy before treatment vs. non-involved skin biopsy, spontaneous resolution of a papule (vehicle signature) was defined as genes differentially expressed between vehicle treated area and a papule biopsy before treatment, and trifarotene signature as genes differentially expressed between trifarotene treated area and a papule biopsy before treatment.

TABLE 1

| Gene Symbol | Gene Name |
|---|---|
| CXCL13 | C-X-C motif chemokine ligand 13 |
| MMP12 | matrix metallopeptidase |
| SPP1 | secreted phosphoprotein 1 |
| ADAMDEC1 | ADAM like decysin 1 |
| MMP13 | matrix metallopeptidase 13 |
| CCNA1 | cyclin A1 |
| ADAM12 | ADAM metallopeptidase domain 12 |
| ITGAX | integrin subunit alpha X |
| BCAT1 | branched chain amino acid transaminase 1 |
| HP | Haptoglobin |
| CBLN2 | cerebellin 2 precursor |
| CTLA4 | cytotoxic T-lymphocyte associated protein 4 |
| KLHL6 | kelch like family member 6 |
| CD80 | CD80 molecule |
| SLAMF7 | SLAM family member 7 |
| SIRPB1 | signal regulatory protein beta 1 |
| SELP | selectin P |
| NCEH1 | neutral cholesterol ester hydrolase 1 |
| GLIS3 | GLIS family zinc finger 3 |
| DMXL2 | Dmx like 2 |
| GLIPR2 | GLI pathogenesis related 2 |
| EVI2B | ecotropic viral integration site 2B |
| NRP2 | neuropilin 2 |
| MMP19 | matrix metallopeptidase 19 |
| FERMT3 | fermitin family member 3 |
| SNAP25 | synaptosome associated protein 25 |
| IRF8 | interferon regulatory factor 8 |
| CYTH4 | cytohesin 4 |
| PAPSS2 | 3′-phosphoadenosine 5′-phosphosulfate synthase 2 |
| P2RY8 | P2Y receptor family member 8 |
| HLA-DPA1 | major histocompatibility complex, class II, DP alpha 1 |
| MSR1 | macrophage scavenger receptor 1 |
| IL2RG | interleukin 2 receptor subunit gamma |
| LOXL2 | lysyl oxidase like 2 |
| PARVG | parvin gamma |
| P2RY10 | P2Y receptor family member 10 |
| GPRIN3 | GPRIN family member 3 |
| RAC2 | Rac family small GTPase 2 |
| CD37 | CD37 molecule |
| PTHLH | parathyroid hormone like hormone |
| INA | internexin neuronal intermediate filament protein alpha |
| CD84 | CD84 molecule |
| PTPRO | protein tyrosine phosphatase receptor type O |
| FNDC3B | fibronectin type III domain containing 3B |
| GLIPR1 | GLI pathogenesis related 1 |
| BIRC3 | baculoviral IAP repeat containing 3 |
| XCL1 | X-C motif chemokine ligand 1 |
| SLC6A14 | solute carrier family 6 member 14 |
| LY86 | lymphocyte antigen 86 |
| ARHGAP9 | Rho GTPase activating protein 9 |
| IL18BP | interleukin 18 binding protein |
| CLEC4A | C-type lectin domain family 4 member A |
| CD69 | CD69 molecule |
| SH2B3 | SH2B adaptor protein 3 |
| TYROBP | TYRO protein tyrosine kinase binding protein |
| GASK1B | golgi associated kinase 1B |
| TRG-AS1 | T cell receptor gamma locus antisense RNA 1 |
| RHOH | ras homolog family member H |
| APEL | Aapelin receptor early endogenous ligand |
| LPXN | leupaxin |
| VCAN | versican |
| CCDC71L | coiled-coil domain containing 71 like |
| PTPN22 | protein tyrosine phosphatase non-receptor type 22 |
| SELPLG | selectin P ligand |
| LINC01224 | — |
| PXMP4 | peroxisomal membrane protein 4 |
| GABRA4 | gamma-aminobutyric acid type A receptor alpha4 subunit |

Core analysis of the acne signature indicated that cellular movement and immune cell trafficking are increased in acne lesions. Pathway analyses showed positive z-scores for TREM1 signaling, crosstalk between dendritic and natural killer cells, leukocyte extravasation signaling, dendritic cell maturation, neuroinflammatory pathways, and pattern recognition receptors involved in recognition of bacteria and viruses (z-score>2.5, P<0.001 for all these pathways). A negative z-score was identified for liver X receptor/retinoid X receptor (LXR/RXR) activation (z-score=−1.78, P<0.001), suggesting retinoid X receptor pathways (and, in turn, keratinocyte differentiation) may be inhibited in acne papules.

IPA analyses of the trifarotene signature showed a positive z-score for LXR/RXR pathways (z-score=1.46 for Trifarotene gene signature), indicating a re-activation during treatment. IPA diseases and functions analyses of the acne gene signature also revealed that cell migration and activation were among the major processes upregulated in acne papules along with a concomitant reduction in neuroinflammation pathway, leukocyte extravasation signaling, cellular movement, and immune cell trafficking (Tables 2 and 3). In line with the IPA analysis, gene set enrichment analyses revealed that Trifarotene treatment indeed downregulates inflammatory modules (“inflammatory response,” “TNFA signaling via NF-kB,” “neutrophil degranulation,” “chemokines and chemokine receptors”) and tissue remodeling (“collagen degradation,” “degradation of the extracellular matrix,” “extracellular matrix organization.” Table 2 shows canonical pathway analysis of trifarotene treated papules. The table represent the canonical pathways with the highest enrichment z-score obtained from the papule signature and from trifarotene signature. All pathways show a reverse enrichment score in the trifarotene signature analysis.

TABLE 2

| | z score | z score |
|---|---|---|
| | Papule | Trifarotene |
| Ingenuity Canonical Pathways | Profile | Profile |
| TREM1 Signaling | 4.123 | −4.796 |
| Crosstalk between Dendritic Cells and Natural | 4.123 | −4.123 |
| Killer Cells | | |
| Neuroinflammation Signaling Pathway | 3.674 | −4.536 |
| Dendritic Cell Maturation | 3.441 | −4.899 |
| Role of NFAT in Regulation of the Immune | 3.357 | −3.771 |
| Response | | |
| Osteoarthritis Pathway | 3.3 | −3.273 |
| Leukocyte Extravasation Signaling | 3.273 | −3.838 |
| Production of Nitric Oxide and Reactive Oxygen | 3.207 | −3.9 |
| Species in Macrophages | | |
| Role of IL-17F in Allergic Inflammatory | 3 | −2.53 |
| Airway Diseases | | |
| Natural Killer Cell Signaling | 2.985 | −3.545 |
| HOTAIR Regulatory Pathway | 2.673 | −2.324 |
| Role of Pattern Recognition Receptors in | 2.53 | −3.873 |
| Recognition of Bacteria and Viruses | | |
| Acute Phase Response Signaling | 2.496 | −3.638 |
| HMGB1 Signaling | 2.309 | −3.606 |
| Fcγ Receptor-mediated Phagocytosis in | 2.309 | −3.464 |
| Macrophages and Monocytes | | |
| Inflammasome pathway | 2.236 | −2.236 |
| T Cell Exhaustion Signaling Pathway | 2.138 | −2.324 |
| IL-17A Signaling in Airway Cells | 2.121 | −2.646 |
| Hepatic Fibrosis Signaling Pathway | 2.041 | −3.889 |
| Tryptophan Degradation to 2-amino-3- | 2 | −2 |
| carboxymuconate Semialdehyde | | |
| NAD biosynthesis II (from tryptophan) | 2 | −2 |
| Th2 Pathway | 1.941 | −1.807 |
| Th1 Pathway | 1.897 | −2.84 |
| LPS/IL-1 Mediated Inhibition of RXR | 1.897 | −1.667 |
| Function | | |
| IL-6 Signaling | 1.732 | −3.051 |
| Systemic Lupus Erythematosus In B Cell | 0.894 | −2.294 |
| Signaling Pathway | | |
| Complement System | 0.816 | −1.897 |
| LXR/RXR Activation | −1.789 | 1.46 |
| Inhibition of Matrix Metalloproteases | −2.53 | 1.897 |

Table 3 shows disease or Functions activation in trifarotene treated papules. This table represents a selection of diseases or functions with the highest activation z-scores, obtained from the papule signature and from the trifarotene signature. All of data show a reverse activation z-score in the Trifarotene of signature analysis.

TABLE 3

| | Activation | Activation |
|---|---|---|
| | z.score | z.score |
| | Papule | Trifarotene |
| Diseases or Functions Annotation | signature | signature |
| Cell movement of leukocytes | 6.973 | −6.976 |
| Cell movement of blood cells | 7.051 | −7.244 |
| Leukocyte migration | 7.202 | −7.285 |
| Activation of cells | 5.611 | −5.508 |
| Cell movement of phagocytes | 6.695 | −7.151 |
| Quantity of leukocytes | 3.968 | −2.579 |
| Cell movement of myeloid cells | 6.714 | −6.93 |
| Activation of leukocytes | 6.041 | −5.289 |
| Proliferation of blood cells | 3.443 | −3.032 |
| Activation of blood cells | 6.281 | −5.514 |
| Quantity of blood cells | 4.309 | −3.022 |
| Proliferation of immune cells | 3.68 | −3.292 |
| Proliferation of mononuclear leukocytes | 3.626 | −3.177 |
| Proliferation of lymphatic system cells | 3.517 | −3.175 |
| Inflammatory response | 7.277 | −7.094 |
| Proliferation of lymphocytes | 3.759 | −3.195 |
| Inflammation of joint | 2.365 | −2.543 |
| Migration of cells | 8.059 | −8.277 |
| Homing of blood cells | 6.948 | −6.815 |
| Chemotaxis of blood cells | 6.991 | −6.617 |
| Homing of leukocytes | 6.948 | −6.814 |
| Binding of blood cells | 6.152 | −6.803 |
| Chemotaxis of leukocytes | 6.991 | −6.617 |
| Cell movement of mononuclear leukocytes | 6.32 | −5.911 |
| Homing of cells | 6.998 | −7.118 |
| Adhesion of blood cells | 6.16 | −6.776 |
| Binding of leukocytes | 6.318 | −6.96 |
| Adhesion of immune cells | 6.364 | −6.945 |
| Cell movement | 8.081 | −8.21 |
| Immune response of leukocytes | 4.414 | −5.255 |
| Chemotaxis | 6.962 | −6.846 |
| Cell movement of granulocytes | 6.538 | −6.539 |
| Chemotaxis of phagocytes | 7.045 | −6.633 |
| Activation of mononuclear leukocytes | 4.986 | −4.237 |
| Cell proliferation of T lymphocytes | 3.29 | −2.957 |
| Activation of lymphatic system cells | 4.83 | −3.911 |
| Chemotaxis of myeloid cells | 6.855 | −6.636 |
| Activation of lymphoid cells | 4.965 | −4.164 |
| Immune response of cells | 5.903 | −6.441 |
| Migration of phagocytes | 5.958 | −5.991 |
| Activation of lymphocytes | 4.894 | −4.094 |
| Degranulation of cells | 3.97 | −4.445 |
| Leukopoiesis | 5.624 | −6.492 |
| Cell movement of neutrophils | 5.985 | −6.35 |
| Migration of mononuclear leukocytes | 5.833 | −5.881 |
| Quantity of lymphatic system cells | 4.256 | −3.034 |
| Cell death of immune cells | 3.177 | −3.66 |
| Cellular infiltration | 4.336 | −3.988 |
| Cell death of blood cells | 2.99 | −3.613 |
| Quantity of mononuclear leukocytes | 3.579 | −2.303 |
| Cell movement of lymphocytes | 6.089 | −5.782 |
| Quantity of lymphoid cells | 3.96 | −2.667 |
| Cellular infiltration by blood cells | 3.962 | −3.72 |
| Lymphocyte migration | 5.942 | −5.932 |
| Cellular infiltration by leukocytes | 4.097 | −3.651 |
| Quantity of lymphocytes | 3.889 | −2.575 |
| Quantity of cells | 4.111 | −3.42 |
| Response of mononuclear leukocytes | 3.661 | −3.434 |
| Cell movement of antigen presenting cells | 5.242 | −5.43 |
| Accumulation of leukocytes | 3.95 | −2.502 |
| Accumulation of blood cells | 4.062 | −2.636 |
| Recruitment of leukocytes | 5.587 | −5.614 |
| Chemotaxis of granulocytes | 6.064 | −5.77 |
| Cellular homeostasis | 6.19 | −6.146 |
| Accumulation of cells | 4.004 | −2.921 |
| Degranulation of phagocytes | 3.067 | −3.602 |
| Chemotaxis of neutrophils | 5.803 | −5.582 |
| Recruitment of cells | 6.026 | −6.218 |
| Activation of phagocytes | 4.865 | −5.069 |
| Recruitment of granulocytes | 4.806 | −5.383 |
| Differentiation of mononuclear leukocytes | 5.484 | −6.486 |
| Hematopoiesis of mononuclear leukocytes | 5.407 | −6.419 |
| Recruitment of myeloid cells | 5.289 | −5.531 |
| Recruitment of neutrophils | 4.536 | −5.008 |
| Response of myeloid cells | 4.106 | −5.29 |
| Hypersensitive reaction | 3.178 | −4.225 |
| Mobilization of Ca2+ | 6.087 | −5.201 |
| Activation of T lymphocytes | 4.83 | −3.731 |
| T cell migration | 5.544 | −4.784 |
| Recruitment of phagocytes | 5.152 | −5.413 |
| Response of phagocytes | 4.614 | −5.241 |
| Cell movement of T lymphocytes | 5.641 | −4.703 |
| Binding of phagocytes | 4.261 | −5.097 |
| Quantity of T lymphocytes | 5.22 | −3.737 |
| Quantity of antigen presenting cells | 3.019 | −2.794 |
| Interaction of mononuclear leukocytes | 5.96 | −6.297 |
| Homeostasis of leukocytes | 5.965 | −6.507 |
| Binding of lymphatic system cells | 5.612 | −5.555 |
| Degranulation of granulocytes | 2.891 | −3.712 |
| Cellular infiltration by myeloid cells | 4.363 | −4.698 |
| Binding of mononuclear leukocytes | 5.811 | −6.006 |
| Interaction of lymphocytes | 5.616 | −5.723 |
| Cellular infiltration by granulocytes | 4.169 | −4.042 |
| Binding of professional phagocytic cells | 4.179 | −5.026 |
| Binding of lymphocytes | 5.536 | −5.477 |
| Lymphopoiesis | 5.553 | −6.509 |
| Lymphocyte homeostasis | 5.881 | −6.304 |
| Homing of mononuclear leukocytes | 5.902 | −5.38 |
| Response of lymphatic system cells | 3.435 | −3.081 |
| Binding of myeloid cells | 3.954 | −4.839 |
| Migration of antigen presenting cells | 4.783 | −4.761 |
| Binding of tumor cell lines | 3.35 | −3.726 |
| Degranulation of neutrophils | 2.433 | −2.804 |
| Advanced malignant tumor | 3.475 | −3.446 |
| Flux of Ca2+ | 3.814 | −2.838 |
| T cell homeostasis | 5.912 | −6.426 |
| Activation of myeloid cells | 4.969 | −5.181 |
| Immune response of phagocytes | 3.831 | −4.658 |
| Migration of myeloid cells | 4.858 | −5.173 |
| Cell movement of dendritic cells | 4.797 | −4.74 |
| T cell development | 5.908 | −6.426 |
| Ion homeostasis of cells | 4.023 | −3.092 |
| Interaction of T lymphocytes | 5.521 | −5.446 |
| Response of lymphocytes | 3.497 | −3.224 |
| Binding of T lymphocytes | 5.459 | −5.203 |
| Adhesion of lymphocytes | 5.47 | −5.487 |
| Cellular infiltration by phagocytes | 4.08 | −4.709 |
| Adhesion of mononuclear leukocytes | 5.738 | −5.99 |
| Chemotaxis of mononuclear leukocytes | 5.623 | −4.929 |
| Cell movement of macrophages | 4.34 | −4.583 |
| Transmigration of cells | 3.504 | −3.498 |
| Stimulation of cells | 5.298 | −5.199 |
| Infiltration by neutrophils | 3.464 | −3.811 |
| Cell-mediated response | 2.328 | −2.605 |
| Cell movement of monocytes | 4.459 | −4.254 |
| Synthesis of reactive oxygen species | 4.329 | −5.497 |
| Binding of granulocytes | 3.272 | −3.934 |
| Adhesion of phagocytes | 3.175 | −3.816 |
| Metabolism of reactive oxygen species | 4.447 | −5.605 |
| Response of granulocytes | 2.003 | −3.203 |
| Adhesion of T lymphocytes | 5.275 | −5.198 |
| Adhesion of granulocytes | 2.841 | −3.499 |

In addition, cellular movement and immune cell trafficking are reduced. Comparison of the profiles generated from an acne lesion with a trifarotene-treated lesion indicated there were 354 genes in common. Notably, all 354 genes were activated or deactivated in a reverse fashion in acne signature compared to trifarotene signature. Analysis of pathways and processes showed reversal of neuroinflammatory pathways, inhibition of matrix metalloproteinase, engagement of LXR/RXR pathway, and inhibition of immune migration. Trifarotene uniquely downregulated 67 genes involved in inflammatory cell infiltration (CXCL13, XCL1) and extracellular matrix reorganization (SPP1/Osteopontin, MMP12, MMP13), as shown in FIG. 3.

Core analysis of spontaneously resolving papules (i.e., vehicle signature) indicated that they include changes in pathways and processes that are similar to those seen with trifarotene therapy. It was observed that 287 genes have changes in expression across acne signature, trifarotene signature, and spontaneously resolved signature and those that are upregulated in acne are downregulated in both trifarotene and spontaneously resolving signatures. These data suggested that the downregulation of previously described acne signature pathways was not necessarily specific to trifarotene treatment. Further 69 genes were observed to overlap between acne and spontaneously resolved signatures. However, 67 genes are uniquely affected and pathway analyses of these genes demonstrated that the treatment with trifarotene is associated with reversal of inflammatory pathways and inhibition of matrix metalloproteinases. The 67 genes which were uniquely affected by trifarotene—these genes did not appear in the spontaneously resolving acne lesion signature (Table 4). Moreover, the treatment also impacted immune cell migration including neutrophil infiltration and T cell migration as shown.

TABLE 4

| | | | Fold | Fold |
|---|---|---|---|---|
| | | | Change | Change |
| | | | L1 vs | R3 vs |
| Symbol | Entrez Gene Name | Affymetrix | L-NI | L1 |
| ADAM12 | ADAM metallopeptidase domain 12 | 226777_at | 3.50 | −3.84 |
| ADAMDEC1 | ADAM like decysin 1 | 206134_at | 6.34 | −6.29 |
| APELA | apelin receptor early endogenous ligand | 1559280_a_at | 2.03 | −2.70 |
| ARHGAP9 | Rho GTPase activating protein 9 | 224451_x_at | 2.10 | −2.14 |
| BCAT1 | branched chain amino acid transaminase 1 | 214452_at | 3.05 | −3.66 |
| BIRC3 | baculoviral IAP repeat containing 3 | 210538_s_at | 2.11 | −2.23 |
| CBLN2 | cerebellin 2 precursor | 242301_at | 2.83 | −2.61 |
| CCDC71L | coiled-coil domain containing 71 like | 229521_at | 2.01 | −2.26 |
| CCNA1 | cyclin A1 | 205899_at | 4.39 | −3.55 |
| CD37 | CD37 molecule | 204192_at | 2.13 | −2.13 |
| CD69 | CD69 molecule | 209795_at | 2.07 | −2.10 |
| CD80 | CD80 molecule | 1554519_at | 2.69 | −4.03 |
| CD84 | CD84 molecule | 230391_at | 2.12 | −2.57 |
| CLEC4A | C-type lectin domain family 4 member A | 221724_s_at | 2.08 | −2.32 |
| CTLA4 | cytotoxic T-lymphocyte associated protein 4 | 236341_at | 2.74 | −2.54 |
| CXCL13 | C-X-C motif chemokine ligand 13 | 205242_at | 20.49 | −23.54 |
| CYTH4 | cytohesin 4 | 219183_s_at | 2.21 | −2.36 |
| DMXL2 | Dmx like 2 | 215761_at | 2.32 | −2.35 |
| EVI2B | ecotropic viral integration site 2B | 211742_s_at | 2.26 | −2.35 |
| FERMT3 | fermitin family member 3 | 223303_at | 2.23 | −2.12 |
| FNDC3B | fibronectin type III domain containing 3B | 229865_at | 2.12 | −2.35 |
| GABRA4 | gamma-aminobutyric acid type A receptor subunit alpha4 | 208463_at | −2.58 | 2.26 |
| GASK1B | golgi associated kinase 1B | 219872_at | 2.05 | −2.30 |
| GLIPR1 | GLI pathogenesis related 1 | 204222_s_at | 2.11 | −2.48 |
| GLIPR2 | GLI pathogenesis related 2 | 225604_s_at | 2.29 | −2.24 |
| GLIS3 | GLIS family zinc finger 3 | 229435_at | 2.34 | −3.07 |
| GPRIN3 | GPRIN family member 3 | 1556698_a_at | 2.15 | −2.27 |
| HLA-DPA1 | major histocompatibility complex, class II, DP alpha 1 | 213537_at | 2.19 | −2.14 |
| HP | haptoglobin | 206697_s_at | 3.00 | −2.99 |
| IL18BP | interleukin 18 binding protein | 222868_s_at | 2.09 | −2.37 |
| IL2RG | interleukin 2 receptor subunit gamma | 204116_at | 2.17 | −2.28 |
| INA | internexin neuronal intermediate filament protein alpha | 204465_s_at | 2.12 | 2.34 |
| IRF8 | interferon regulatory factor 8 | 204057_at | 2.22 | −2.02 |
| ITGAX | integrin subunit alpha X | 210184_at | 3.22 | −2.85 |
| KLHL6 | kelch like family member 6 | 1555275_a_at | 2.70 | −3.05 |
| LINC01224 | — | 233142_at | −2.05 | 3.51 |
| LOXL2 | lysyl oxidase like 2 | 202998_s_at | 2.16 | −2.18 |
| LPXN | leupaxin | 216250_s_at | 2.03 | −2.25 |
| LY86 | lymphocyte antigen 86 | 205859_at | 2.10 | −2.18 |
| MMP12 | matrix metallopeptidase 12 | 204580_at | 19.41 | −11.13 |
| MMP13 | matrix metallopeptidase 13 | 205959_at | 4.86 | −4.30 |
| MMP19 | matrix metallopeptidase 19 | 204575_s_at | 2.24 | −2.71 |
| MSR1 | macrophage scavenger receptor 1 | 214770_at | 2.18 | −2.45 |
| NCEH1 | neutral cholesterol ester hydrolase 1 | 225847_at | 2.41 | −2.70 |
| NRP2 | neuropilin 2 | 223510_at | 2.25 | −2.46 |
| P2RY8 | P2Y receptor family member 8 | 229686_at | 2.20 | −2.23 |
| P2RY10 | P2Y receptor family member 10 | 236280_at | 2.16 | −2.05 |
| PAPSS2 | 3′-phosphoadenosine 5′-phosphosulfate synthase 2 | 203060_s_at | 2.21 | −2.60 |
| PARVG | parvin gamma | 223562_at | 2.16 | −2.23 |
| PTHLH | parathyroid hormone like hormone | 211756_at | 2.13 | −2.55 |
| PTPN22 | protein tyrosine phosphatase non-receptor type 22 | 236539_at | 2.01 | −2.27 |
| PTPRO | protein tyrosine phosphatase receptor type O | 208121_s_at | 2.12 | −2.26 |
| PXMP4 | peroxisomal membrane protein 4 | 238746_at | −2.19 | 2.14 |
| RAC2 | Rac family small GTPase 2 | 207419_s_at | 2.13 | −2.03 |
| RHOH | ras homolog family member H | 204951_at | 2.04 | −2.57 |
| SELP | selectin P | 206049_at | 2.42 | −2.10 |
| SELPLG | selectin P ligand | 209879_at | 2.01 | −2.06 |
| SH2B3 | SH2B adaptor protein 3 | 203320_at | 2.07 | −2.26 |
| SIRPB1 | signal regulatory protein beta 1 | 206934_at | 2.51 | −2.45 |
| SLAMF7 | SLAM family member 7 | 219159_s_at | 2.64 | −2.49 |
| SLC6A14 | solute carrier family 6 member 14 | 219795_at | 2.10 | −2.04 |
| SNAP25 | synaptosome associated protein 25 | 202508_s_at | 2.23 | −2.40 |
| SPP1 | secreted phosphoprotein 1 | 1568574_x_at | 12.63 | −28.23 |
| TRG-AS1 | T cell receptor gamma locus antisense RNA 1 | 239237_at | 2.05 | −2.33 |
| TYROBP | transmembrane immune signaling adaptor TYROBP | 204122_at | 2.06 | −2.39 |
| VCAN | versican | 215646_s_at | 2.02 | −2.43 |
| XCL1 | X-C motif chemokine ligand 1 | 206366_x_at | 2.10 | −2.02 |

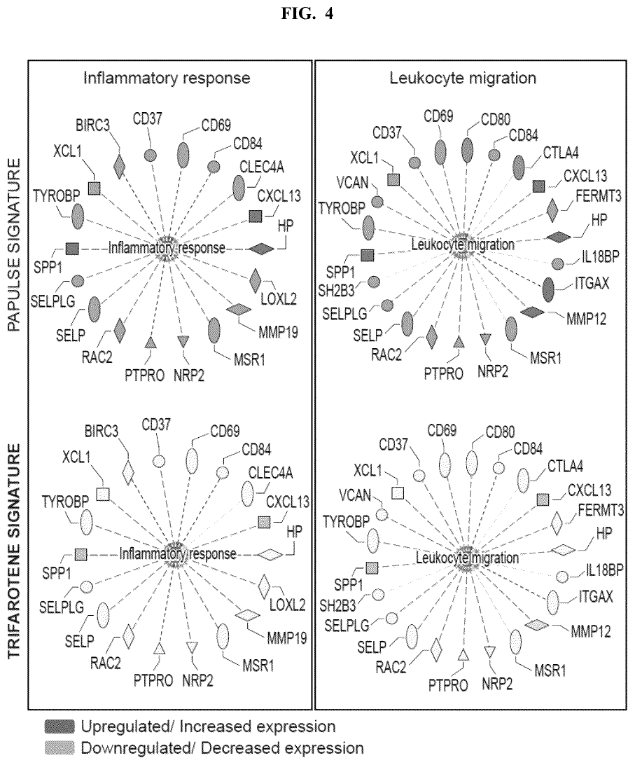

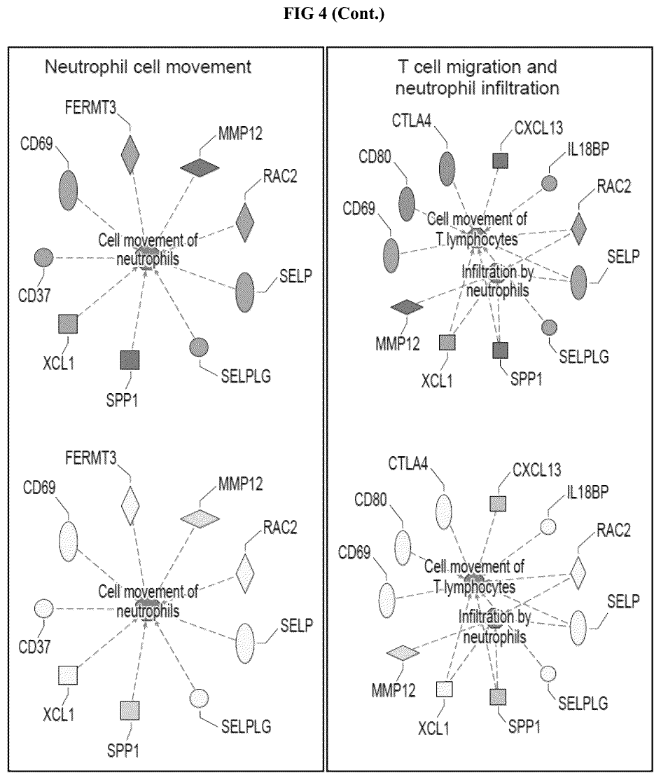

As seen in FIG. 3, majority of the 67 genes were upregulated in acne lesions (as compared to non-involved skin) and were concomitantly downregulated by trifarotene treatment only. The most significantly down-regulated genes included the chemokines CXCL13 (Fold-Change FC=−23.5, FDR=0.0032) and XCL1 (FC=−2.02, FDR=0.013), the phosphoglycoprotein osteopontin (SPP1, FC=−28.2, FDR=0.0022) and the matrix metalloproteinases MMP12 and MMP13 (FC=−11.13, FDR=0.061; FC=−4.3, FDR=0.029, respectively). These genes influence inflammatory cell infiltration (including CXCL13, XCL1, and SPP1/osteopontin) and extracellular matrix reorganization (MMP12 and MMP13) (Table 4). IPA analysis of the 67 genes (FIG. 4) revealed that an upregulation of pathways associated with inflammatory response, leukocyte migration, neutrophil movement, and T cell migration seen in the papule was reversed following trifarotene treatment.

Of the 67 genes regulated by trifarotene, there were only four genes upregulated: long intergenic non-protein coding RNA 1224 (LINC01224), alpha-internexin (INA), gamma-aminobutyric acid receptor subunit alpha-4 (GABRA4) and peroxisomal membrane protein 4 (PXMP4) (Table 4). There is limited information on the role of these genes on inflammation and skin homeostasis. LINC01224 has been recently described to promote cell proliferation and survival. GABRA4 and INA play a role in the nervous system with GABA4 being a major inhibitory neurotransmitter and INA is a neuronal specific intermediate filament playing a role in neuronal development. The impact of upregulation of these genes following trifrotene treatment will be of great interest. To identify if these pathways were specific to trifarotene treatment, the 69 genes uniquely regulated in spontaneously resolved lesions were analyzed. The major downregulated genes in spontaneously resolved papules included inflammation related genes like chemokine CXCL10 and beta defensin 103 (DEFB103A/DEFB103B), transcobalamin-1 (TCN1), and epithelial-related genes including S100A7A, S100A9, SPRR3, SERPINB3, and SERPINB4. Pathway analysis of these genes revealed that while spontaneous resolution of lesions is associated with decreasing inflammation, there was no impact on extracellular matrix organization in contrast to what was observed with trifarotene. The relative roles of CXCL10 and CXCL13 on recruitment of specific T cell subsets in the context of acne pathogenesis will be further investigated.

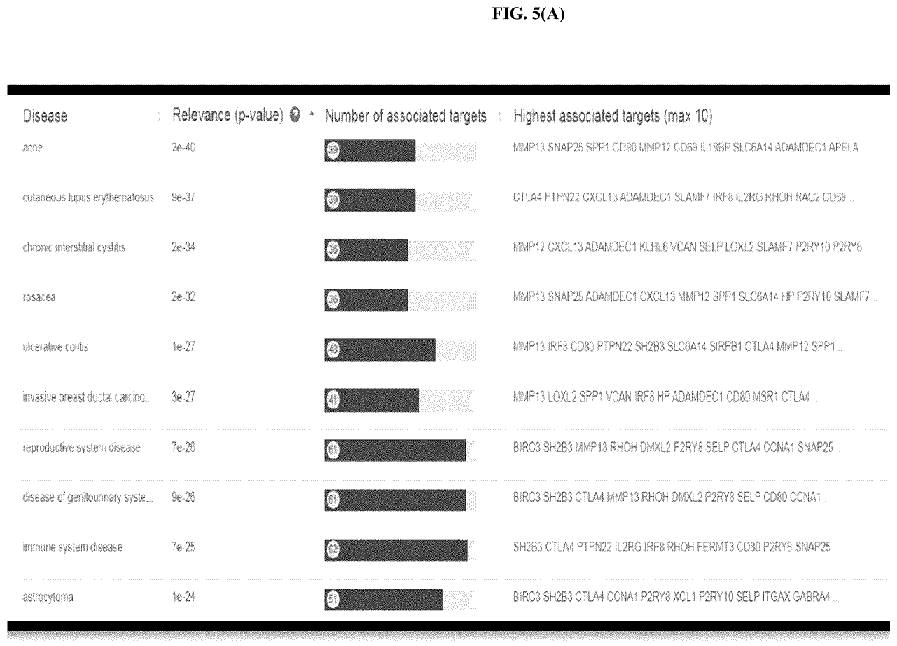

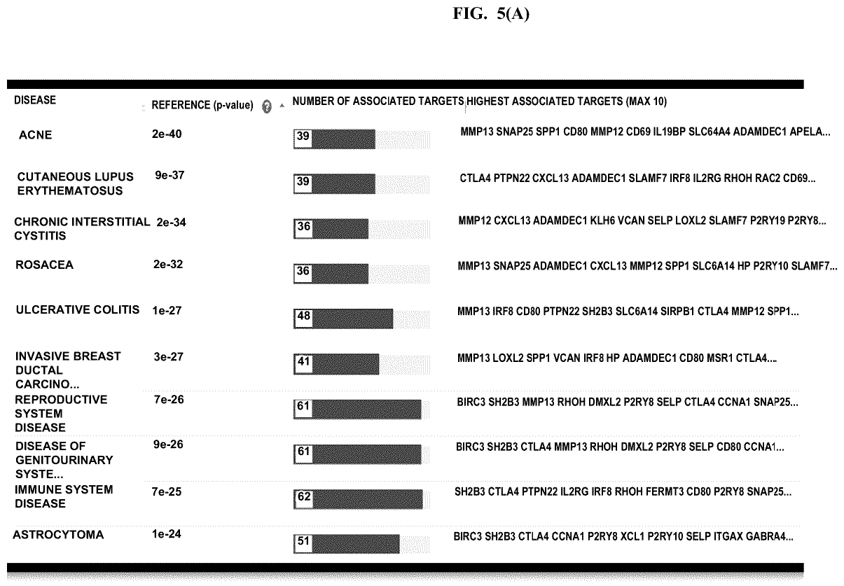

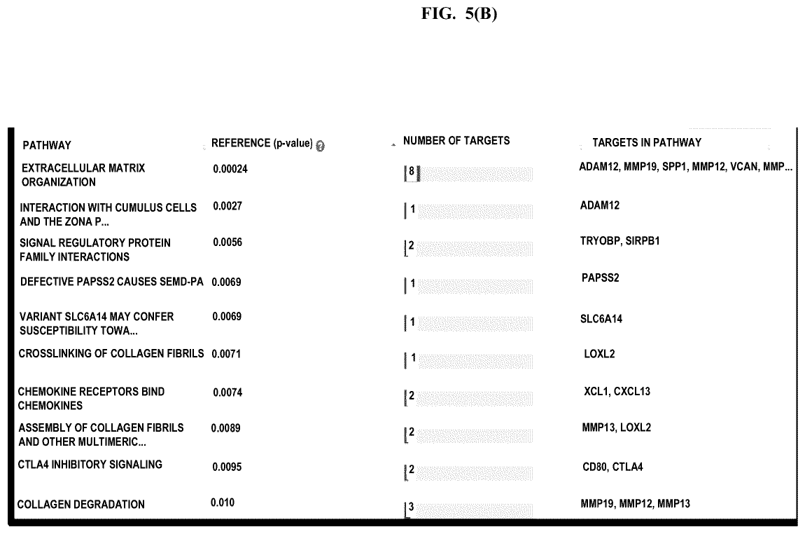

Acne lesions exhibit increased expression of genes associated with inflammation and cell infiltration. There also appears to be a downregulation of LXR/RXR signalling. The treatment appears to reverse these processes, as a total of 354 genes upregulated in acne are reversed by retinoic acid receptor compounds. Sixty-seven genes involved in inflammatory cell infiltration (CXCL13, XCL1) and extracellular matrix reorganization (SPP1/Osteopontin, MMP12, MMP13) are uniquely downregulated. Two independent systems biology approaches are used here to better characterize this list of 67 genes: Ingenuity Pathway Analysis and Open Targets. Results of the open target analyses are depicted in FIGS. 5A and 5B. In the Ingenuity analysis, the most significant processes identified were inflammatory response, leukocyte migration, Neutrophil cell movement and T cell migration and neutrophil infiltration, suggesting that trifarotene may reverse some inflammatory processes of the papule. The Open Targets results illustrate that acne and immune system diseases were among top processes regulated by trifarotene specific genes. Extracellular matrix remodeling is the most represented target pathway through the modulation of 8 genes: MMP12, MMP13, MMP19, ADAM12, ADAMDEC1, SSP1 and VCAM. Two targets were imputed to chemokine signaling: C-X-C motif chemokine ligand 13 (CXCL13) and X-C motif chemokine ligand (XCL1). Thus, trifarotene has a unique action in acne treatment by acting both on epidermal and immune components of acne pathogenesis.

In particular, a notable gene strongly down regulated was chemokine (C-X-C motif) ligand 13 (CXCL13), a major chemoattractant of B cells and a regulator of humoral immune responses. CXCL13 is a small chemokine belonging to the CXC chemokine family and is selectively chemotactic for B cells belonging to both the B-1 and B-2 subsets, and elicits its effects by interacting with chemokine receptor type 5 (CXCR5). CXCL13 and its receptor CXCR5 control the organization of B cells within follicles of lymphoid tissues. In T lymphocytes, CXCL13 expression is thought to reflect a germinal center origin of the T cell, particularly a subset of T cells called follicular helper T cells (or TFH cells). This suggests that trifarotene may have an effect on the adaptive immune response in the perilesional skin of acne patients. Other major genes which have reduced expression or are specifically inhibited include secreted phosphoproteins, such as SPP1, that bind to various types of calcium-based biominerals, and members of the matrix-metalloproteinase family, such as MMP12 and MMP13, that play a role in the extra-cellular matrix reorganization and tissue remodeling, processes which may actively be involved in acne. Other examples of genes which may have reduced expression are listed in Table 1. Comparison of skin post resolution shows that the treatment also promotes epidermal differentiation and keratinization processes. Altogether, these results show that retinoic acid (RA) or a synthetic RAR agonist, such as trifarotene has a unique action in acne treatment by acting on epidermal and immune components of acne pathogenesis.

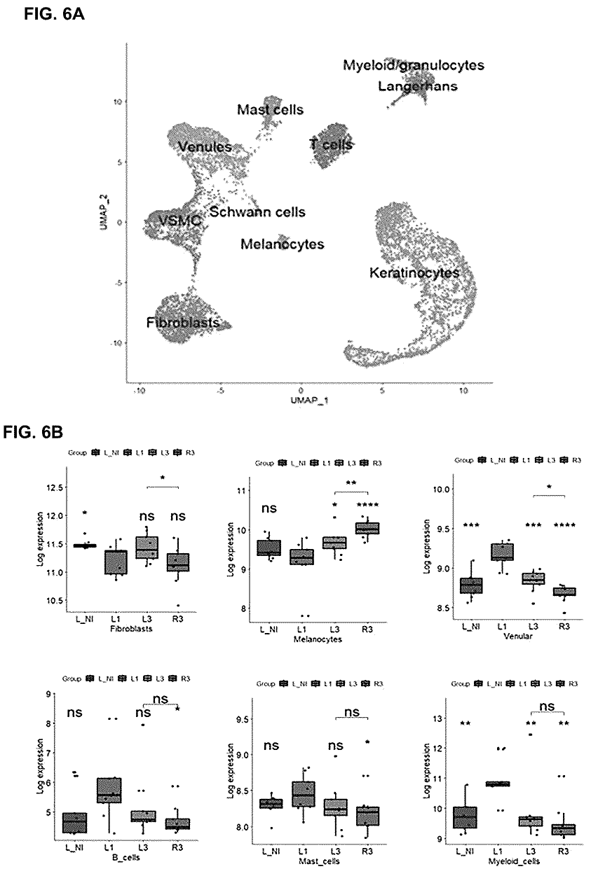

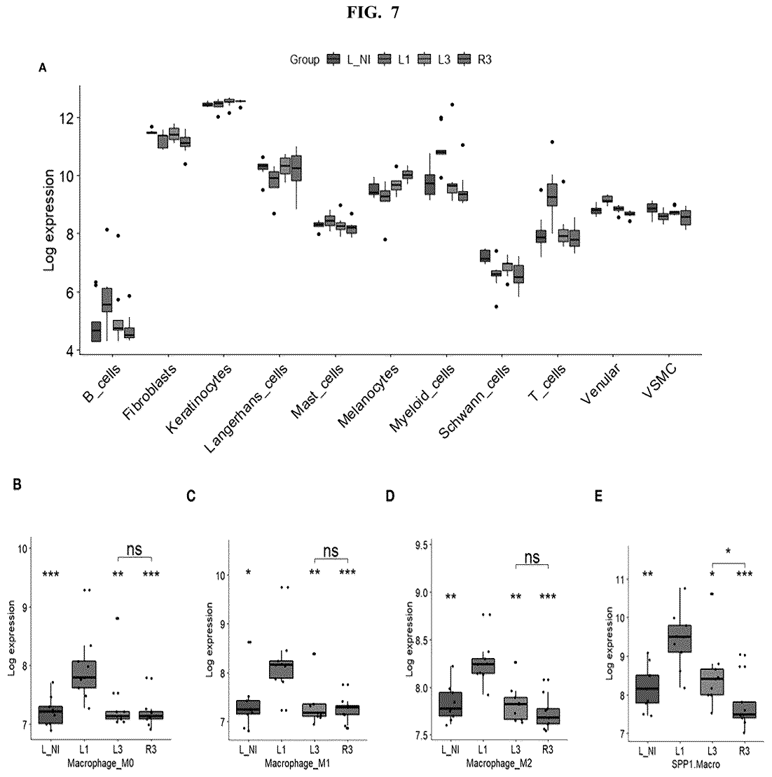

To further investigate the effects of trifarotene on the various cell types present in skin biopsies, specific gene signatures through the re-analysis of publicly available single cell RNAseq data from healthy and acne skin biopsies were obtained (GSE150672, Hughes et al. Second-strand synthesis-based massively parallel scRNA-Seq reveals cellular states and molecular features of human inflammatory skin pathologies. Immunity. 53:878-94, 2020). 10 major cell types were identified, as presented in the UMAP visualization (FIG. 6; ns, non-significant; p<0.05; **p<0.01; ***p<0.0001.) The repartition of normal and acne cells was balanced in each group. From this analysis, marker genes for each cell type were obtained, and then a cell-type specific gene expression score in each of our study samples was generated. The distribution of these scores is represented in the boxplots in FIG. 7 (In FIG. 7, *Represent the statistical significance of the difference in gene expression when comparing non-involved skin (L_NI), with acne papule (L1), spontaneously resolved papule (L3), or Trifarotene treated skin (R3). While expression of each class of macrophages is reduced when compared to papule signature, only SPP1 activated macrophages show a significant difference when compared to spontaneous resolution of acne papule. For each sample, an expression score was derived from selected marker genes for each cell type. Changes in expression in each clinical group were represented using boxplots. ns, non-significant; *p<0.05; **p<0.01; ***p<0.0001.) There was a significant increase in immune cells, such as B cells, T cells, and myeloids/granulocytes in acne (L1) compared to non-involved skin. Upon resolution of the acne papule over time, with or without treatment with trifarotene, this over-expression of immune cells decreases. A significant reduction in expression of venular cell genes, and a significant increase in melanocytes, keratinocytes, and myeloid cells was also observed (FIG. 6, FIG. 7).

Cell type scoring analysis results showed that all subsets of macrophages, including the relatively newly identified SPP1/MERTK activated (SPP1⁺) macrophages, were increased in lesional skin and tended back toward normal levels with spontaneous lesion resolution or trifarotene-induced lesion resolution. As shown in FIG. 7, M0, M1 and M2 macrophages decreased with papule resolution but were not significantly different in spontaneously resolved or trifarotene treated biopsies. However, trifarotene treatment significantly reduced frequencies of SPP1⁺ macrophages vs. spontaneously resolved acne papules (P=0.040).

The present technology highlights a specific gene expression profile, comprised of 67 genes uniquely driven by trifarotene treatment with most of these genes known to affect immune responses as well as extracellular matrix reorganization. Interestingly, those genes were modulated in an opposite direction as compared to the untreated papule signature suggesting reversal of some inflammatory processes within the papule. MMP12 and MMP13, CXCL13, XCL1, and SPP1/Osteopontin, were observed to be the most dominantly regulated. Pathways impacted by trifarotene included pro-inflammatory responses, neutrophil chemotaxis, and T cell migration.

To determine whether expression changes of the 67 unique trifarotene-affected genes was associated with a change in cellular signature, markers associated with specific cell types were studied. Increased B cell and macrophage frequencies were found in acne lesions. Macrophages have both protective and pathogenic functions in skin and have classically been divided into subsets (M1=pro-inflammatory and antimicrobial; M2=anti-inflammatory and wound healing, and M0=naïve macrophages). While wound healing is driven by M2 macrophages, the newly described SPP1+ subset have been implicated in driving fibrosis, at least in the lungs. The increased frequencies of this latter population in acne papules suggest that these cells potentially drive fibrosis and production of MMPs, growth factors, and cytokines in acne lesions.

Soluble osteopontin (OPN) is a multi-functional cytokine found elevated in psoriasis and other inflammatory diseases. It binds to integrins and CD44 on immune cells and plays a major role in cell adhesion, migration, and modulation of both Th1 and Th17 responses. OPN may have a role in acne via these effects.

The chemokine CXCL13 and its receptor, CXCR5, play a central role in driving humoral immunity during infection and vaccine responses. Originally identified as a Bcell chemoattractant, CXCL13 exerts important functions in lymphoid neogenesis, and has been widely implicated in the pathogenesis of several autoimmune diseases and inflammatory conditions, as well as in lymphoproliferative disorders. In line with the CXCL13 expression data, cellular analysis not only showed increased B cells in acne papules, but also that trifarotene treatment had a greater impact in reducing B cell frequencies vs. spontaneous resolution. B cells have been implicated in hidradenitis suppurativa, where they are thought to amplify inflammatory response. Similarly, in acne, increased numbers of B cells have been correlated with higher severity of disease. Gene expression and immunohistochemistry analyses showed a very similar immune response in 48-hold papules in patients prone or not prone to scarring, characterized by elevated numbers of T cells, neutrophils and macrophages. However, the immune response only persisted in patients prone to scarring in 3-week-old papules, and was characterized by an important B-cell infiltrate.

MMPs are involved in tissue destruction and have a major role in scar formation and can mediate innate immune responses. In healthy skin, MMPs play an essential role in regulating the skin matrix. C. acnes induces production of various MMPs, and extracellular matrix remodeling regulated by MMPs is thought to be a part of the pathogenesis of acne. The transcription factor activator protein-1 (AP-1), which regulates the expression of several MMPs has been shown to be upregulated in acne lesions. Targeting MMPs presents a potential way to minimize scar development and abnormal skin remodeling. An increase in melanocytes occurred in both spontaneous and treated resolved acne lesions, and was greater in the trifarotene treated samples. In addition, there is a trend toward a decrease of venular cells following trifarotene treatment, which may indicate a decrease in cells of the vasculature or inflammatory cell recruitment. The results presented here indicate that trifarotene, a retinoic acid receptor gamma (RARg) selective agonist, regulates several unique genes and pathways.

### Methods

In accordance with one aspect, provided are method for treatment of a skin disorder in a subject in need thereof, the method comprising, consisting of, or consisting essentially of administering to the subject, a pharmaceutical composition, wherein said pharmaceutical composition reduces the expression of one or more genes associated with reversal of inflammatory pathways or matrix reorganization. Examples of such genes include, but are not limited to CXCL13, XCL1, SPP1, MMP12 and MMP13. In accordance with one, provided are methods of treating a skin disorder in a subject, the method comprising, consisting of, or consisting essentially of administering a pharmaceutical composition that reduces the expression of CXCL13. In accordance with a second aspect, provided are methods of treating a skin disorder in a subject, the method comprising, consisting of, or consisting essentially of administering a pharmaceutical composition that reduces the expression of MMP12 and/or MMP13. In accordance with a third aspect, provided are methods of treating a skin disorder in a subject, the method comprising, consisting of, or consisting essentially of administering a pharmaceutical composition that reduces the expression of a combination of CXCL13 and MMP12 and/or MMP13. In accordance with a fourth aspect, provided are methods of treating a skin disorder in a subject, the method comprising, consisting of, or consisting essentially of administering a pharmaceutical composition, wherein said pharmaceutical composition reduces the expression of one or more genes selected from CXCL13, XCL1, SPP1, MMP12 and MMP13.

In accordance with another aspect, provided are method for treatment of a skin disorder in a subject in need thereof, the method comprising, consisting of, or consisting essentially of administering to the subject, a pharmaceutical composition, wherein said pharmaceutical composition inhibits the expression or activity of one or more genes associated with reversal of inflammatory pathways or matrix reorganization. In accordance with one or more embodiments, there are provided methods for treatment of a skin disorder in a subject, the method comprising administering to said subject a pharmaceutical composition that inhibits CXCL13 expression. In accordance with one or more embodiments, there are provided methods for treatment of a skin disorder in a subject, the method comprising administering to said subject a pharmaceutical composition that inhibits MMP12 and/or MMP13 expression. In accordance with one or more embodiments, there are provided methods for treatment of a skin disorder in a subject, the method comprising administering to said subject a pharmaceutical composition exhibiting combined inhibition of CXCL13 and MMP12 and/or MMP13 expression. In one or more embodiments, the pharmaceutical composition comprises a therapeutically effective amount of a pharmaceutical agent and a pharmaceutically acceptable carrier. In one or more embodiments, the pharmaceutical agent reduces the expression of one or more genes selected from CXCL13, XCL1, SPP1, MMP12 and MMP13. In accordance with one or more embodiments, there are provided methods for treatment of a skin disorder in a subject in need thereof, the method comprising administering to a subject in need thereof, a pharmaceutical composition, wherein said pharmaceutical composition reduces the expression of one or more genes selected from CXCL13, XCL1, SPP1, MMP12 and MMP13.

The skin disorders may include dermatological conditions such as inflammatory skin disorders, skin cancers, disorders of increased cell turnover (e.g. psoriasis), photoaging, and skin wrinkles. In one or more embodiments, the skin disorder may include, but is not limited to, acne, psoriasis, ichthyos, ichthyosiform states and palmoplantar hyperkeratosis. In one or more embodiments, the skin disorder is acne. In one or more embodiments, the skin disorder is acne vulgaris. In one or more embodiments, the skin disorder is acne vulgaris of the face and/or trunk.

The success rate of the treatment disclosed herein may be based on the IGA and PGA outcome (percentage of subjects “clear” and “almost clear” and with at least a 2-grade change from baseline) and/or the absolute and percentage change from baseline lesion counts on the face and/or trunk. The lesion counts consider the two major types of acne lesions: non-inflammatory and inflammatory. Non-inflammatory lesions of acne are the open (blackheads) or closed (whiteheads) comedones. Inflammatory lesions are divided into papules, pustules, and nodules/nodulocystic lesions, depending on the severity and location of the inflammation within the dermis. Both the absolute and percentage reduction in total number of inflammatory and/or non-inflammatory lesions on the face and/or trunk from the first day of treatment (baseline) to the end of the treatment may be used to assess the success rate. In one or more embodiments, the treatment may include reduction in inflammatory and/or noninflammatory lesion counts of the subject.

In particular embodiments, the subject has at least 20 inflammatory lesions and 25 non-inflammatory lesion counts on the face at screening and baseline. In particular embodiments, the subject has at least 20 inflammatory lesions and 20 non-inflammatory lesions but no more than 100 non-inflammatory lesion counts on the trunk (shoulders, upper back and chest, anterior chest) at screening and baseline. In one or more embodiments, the treatment may include about 10% reduction in inflammatory and/or non-inflammatory lesion count on the face and/or trunk in a subject, including, but not limited to, greater than about 15%, greater than about 20%, greater than about 30%, greater than about 40% or greater than about 50% reduction in inflammatory and/or non-inflammatory lesion count.

In one or more embodiment, the treatment may include from about 5% to about 99% reduction in inflammatory and/or non-inflammatory lesion count in a subject, including, but not limited to, about 10% to about 90%, about 15% to about 85%, about 20% to about 80%, about 25% to about 75%, about 30% to about 70%, about 35% to about 65%, about 40% to about 60%, about 45% to about 50% reduction in inflammatory and/or non-inflammatory lesion count on the face and/or trunk, or any range including and/or in-between any two of these values. In one or more embodiments, the treatment includes about 20% to about 60% reduction in inflammatory and/or non-inflammatory lesion count on the face and/or trunk after 12 weeks of treatment. In one or more embodiments, the treatment includes about 30% to about 50% reduction in inflammatory and/or non-inflammatory lesion count on the face and/or trunk after 12 weeks of treatment.

The pharmaceutical composition may include a pharmaceutical agent and a pharmaceutically acceptable carrier. Suitable pharmaceutical agent includes compounds which reduce the expression of one or more genes associated with reversal of inflammatory pathways or matrix reorganization. In some embodiments, pharmaceutical agent includes compounds which reduce the expression or inhibit the expression or activity of one or more genes associated with reversal of inflammatory pathways or matrix reorganization. In one or more embodiments, the pharmaceutical agent is one that reduces the expression of chemokine (C-X-C motif) ligands such as e.g., CXCL13. In one or more embodiments, the pharmaceutical agent is that reduces the expression of the secreted phosphoprotein genes such as e.g., SPP1. In one or more embodiments, the pharmaceutical agent is that reduces the expression of genes from the metalloproteinase family such as e.g., MMP12 and MMP13. In some embodiments, pharmaceutical agent includes compounds which inhibit the expression or activity of one or more genes selected from CXCL13, XCL1, SPP1, MMP12 and MMP13. Suitable pharmaceutical agents which reduce the expression of these genes, optionally which have such inhibitory activity and which can be used in the treatment of acne include, for example, retinoids, including compounds which are retinoid acid or a synthetic retinoic acid receptor (RAR) agonists. In one or more embodiments, the pharmaceutical agent is retinoic acid or a synthetic RAR agonist. In one or more embodiments, the pharmaceutical agent includes trifarotene or an equivalent thereof. In one or more embodiments, the pharmaceutical agent does not include trifarotene or a pharmaceutically acceptable salt thereof. In one or more embodiments, the pharmaceutical agent is not trifarotene. In one or more embodiments, the pharmaceutical agent is retinoic acid or a synthetic RAR agonist other than trifarotene.

An effective amount can be administered in one or more administrations, applications or dosages. Such delivery is dependent on a number of variables including the time period for which the individual dosage unit is to be used, the bioavailability of the therapeutic agent, the route of administration, etc. The pharmaceutical compositions or formulations may be delivered to the subject by various routes of administration, e.g., by topical administration, transdermal administration, oral administration, by nasal administration, rectal administration, subcutaneous injection, intravenous injection, intramuscular injection, or intraperitoneal injection. It is understood, however, that specific dose levels of the therapeutic agents of the present disclosure for any particular subject depends upon a variety of factors including the activity of the specific compound employed, the age, body weight, general health, sex, and diet of the subject, the time of administration, the rate of excretion, the drug combination, skin absorption for topical compositions, systemic absorption in general for topical and oral compositions, and the severity of the particular disorder being treated and form of administration. Treatment dosages generally may be titrated to optimize safety and efficacy. The dosage can be determined by a physician and adjusted, as necessary, to suit observed treatment effects and to manage and adapt to retinoid dermatitis.

In one or more embodiments, creams, ointments, foams or lotions which includes from about 1 μg/g to about 100 μg/g of the pharmaceutical agent is topically administered to the subject, including, but not limited to, from about 5 μg/g to about 95 μg/g, about 10 μg/g to about 90 μg/g, about 15 μg/g to about 85 μg/g, about 20 μg/g to about 80 μg/g, about 25 μg/g to about 75 μg/g, about 30 μg/g to about 70 μg/g, about 35 μg/g to about 65 μg/g, about 40 μg/g to about 60 μg/g, about 45 μg/g to about 55 μg/g, about 46 μg/g to about 54 μg/g, about 47 μg/g to about 53 μg/g, about 48 μg/g to about 52 μg/g, or about 49 μg/g to about 51 μg/g. In certain embodiments, a cream which includes from about 1 μg/g to about 100 μg/g of the pharmaceutical is topically administered to the subject. In certain embodiments, a cream which includes about 50 μg/g of the pharmaceutical agent is topically administered once daily to the affected area of the subject.

In one or more embodiments, the effective amount of the pharmaceutical agent ranges from about 0.0001 weight percent to about 0.1 weight percent, about 0.001 weight percent to about 0.5 weight percent, about 0.005 weight percent to about 1 weight percent, about 0.01 weight percent to about 1.5 weight percent, or about 0.1 weight percent to about 10 weight percent. In particular embodiments, the effective amount of the pharmaceutical agent is about 0.0001 weight percent, about 0.005 weight percent, about 0.1 weight percent, about 1 weight percent, about 2 weight percent, or about 2.5 weight percent, or any range including and/or in-between any two of these values, and/or as needed based on the appearance of symptoms of the skin disorder. In particular embodiments, the effective amount of the pharmaceutical agent is a 2 g flat dose (e.g. in cream form). In one or more embodiments, the pharmaceutical agent is administered by a topical route. In one or more embodiments, the pharmaceutical agent is administered by applying a thin layer enough to cover the area being treated.

In one or more embodiments, the pharmaceutical composition described herein is administered thrice daily, twice daily, once daily, every other day, twice per week, three times per week, four times per week, five times per week, six times per week, once per week, once every two weeks, once every three weeks, once every four weeks, once every five weeks, once every six weeks, once every seven weeks, once every eight weeks, once every nine weeks, once every 10 weeks, once every 11 weeks, once every 12 weeks, twice per year, once per year, or any range including and/or in-between any two of these values, and/or as needed based on the appearance of symptoms of the skin disorder. In one or more embodiments, the pharmaceutical composition is administered once daily.

The treatments have a variable duration, depending on the patient and the severity of the facial and/or acne. The treatment period may thus run from several days to several years. In one or more embodiments, the duration of treatment is about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about one week, about 2 weeks, about 3 weeks, about 4 weeks, about 5 weeks, about 10 weeks, about 20 weeks, about 30 weeks, about 36 weeks, about 40 weeks, about 48 weeks, about 50 weeks, about one year, about two years, about three years, about four years, about five years, or any range including and/or in-between any two of these values, and/or as needed based on the appearance of symptoms of the skin disorder. In one or more embodiments, duration of treatment is about 10 days to about 35 days, about 25 days to about 30 days, about 12 to about 48 weeks, or about 24 to about 36 weeks.

In accordance with another aspect, provided are methods of modulating an adaptive immune response in a subject to effect a treatment of a skin disorder, comprising administering to the subject a therapeutically effective amount of a trifarotene or an equivalent thereof and a pharmaceutically acceptable carrier. In one or more embodiments, the skin disorder is acne vulgaris. In one or more embodiments, the skin disorder is acne vulgaris of the face and/or trunk, including moderate acne vulgaris of the face and trunk.

In accordance with one aspect, provided are methods of preventing, attenuating, alleviating or treating inflammatory response associated with skin disorders in a subject, comprising topically administering to the subject a therapeutically effective amount of a pharmaceutical agent and a pharmaceutically acceptable carrier. Suitable pharmaceutical agents are described herein and include compounds which inhibit the activity of or reduce the expression of one or more genes associated with reversal of inflammatory pathways or matrix reorganization such as e.g., CXCL13, XCL1, SPP1, MMP12 and MMP13. In one or more embodiments, the skin disorder is acne vulgaris and the method comprises treating acne vulgaris. In one or more embodiments, the method decreases inflammation in the skin of the subject.

In accordance with another aspect, provided are methods of antagonizing CXCR5 receptor activity in a patient suffering from acne vulgaris, consisting of administering to the patient in need thereof a therapeutically effective amount of a pharmaceutical agent which inhibits the activity of or reduces the expression of one or more genes associated with reversal of inflammatory pathways such as e.g., CXCL13.

In accordance with another aspect, provided are methods monitoring or determining efficacy of a skin disorder treatment, including an acne treatment in a subject, the method comprising determining in a sample from said subject the expression of one or more genes selected from CXCL13, XCL1, SPP1, MMP12 and MMP13. In one or more embodiments, the acne is acne vulgaris. In one or more embodiments, the subject is treated with a pharmaceutical composition, which includes trifarotene or an equivalent thereof. In one or more embodiments, a decrease or an increase in the expression of one or more genes selected from CXCL13, XCL1, SPP1, MMP12 and MMP13 in the sample relative to the controls is used to determine the efficacy of the acne treatment. The present technology also provides methods for diagnosing or detecting inflammatory response in a subject suffering from a skin disorder, e.g., acne vulgaris, comprising determining in a sample from said subject, a decrease or an increase in the expression of one or more genes selected from CXCL13, XCL1, SPP1, MMP12 and MMP13 in the sample relative to the controls is used to determine the efficacy of the acne treatment. The controls can be subjects who have not undergone the treatment with the pharmaceutical agent.

### Pharmaceutical Compositions

Provided herein are pharmaceutical compositions for use in the methods described herein, the composition comprising, consisting of, or consisting essentially of a pharmaceutical agent. In one or more embodiments, the pharmaceutical agent is a retinoid, or an equivalent thereof. In one or more embodiments, the pharmaceutical agent is a retinoid, such as trifarotene or an equivalent thereof. In one or more embodiments, the pharmaceutical agent includes trifarotene or a pharmaceutically acceptable salt thereof. In one or more embodiments, the pharmaceutical composition may include a therapeutically effective amount trifarotene or an equivalent thereof and a pharmaceutically acceptable carrier. In one aspect, the present technology provides a pharmaceutical composition for alleviating inflammation associated with skin disorders comprising trifarotene or an equivalent thereof and a pharmaceutically acceptable carrier.

In one aspect, the present technology provides a pharmaceutical composition including a pharmaceutical agent and a pharmaceutically acceptable carrier. One of ordinary skill in the art will appreciate that the term “effective amount” may not require successful treatment be achieved in a particular individual. In some embodiments, effective amount may be that amount that provides a particular desired pharmacological response in a significant number of subjects when administered to patients in need of such treatment. In some embodiments, an effective amount may be formulated and/or administered in a single dose. In other embodiments, an effective amount may be formulated and/or administered in a plurality of doses, for example, as part of a dosing regimen. In one or more embodiments, the effective amount may be an effective amount for treating a skin disorder (e.g., acne vulgaris), for modulating an adaptive immune response, for preventing, attenuating, alleviating or treating inflammatory response associated with skin disorders, for antagonizing CXCR5 receptor activity, for monitoring or determining efficacy of an acne treatment or for diagnosing or detecting inflammatory response in a subject.

It is recognized that pharmaceutical compositions may be provided in the form of a cream or a foam which may be dispensed from a tube or a pump. In some embodiments, the pharmaceutical compositions may be packed as a 50 mL bottle with pump and over cap. In some embodiments, the pharmaceutical composition provided herein includes an effective amount of an active ingredient, which includes an amount (and/or a concentration) sufficient so that a single actuation of a pump containing the composition produces an effective amount. In some embodiments, more than one actuation of the pump may be required to produce an effective amount of active ingredient. For example, one pump actuation may include an amount of the pharmaceutical composition which is enough to cover the face (i.e., forehead, cheeks, nose, and chin), two actuations of the pump may include an amount of the pharmaceutical composition which is enough to cover the upper trunk (i.e., reachable upper back, shoulders and chest). In some embodiments, one additional pump actuation may be used for application to the middle and lower back if acne is present.

The pharmaceutical compositions described herein may contain various carriers or excipients known to those skilled in the art. Such excipients and carriers are described, for example, in “Remington's Pharmaceutical Sciences” Mack Pub. Co., New Jersey (1991), which is incorporated herein by reference. Suitable carriers or excipients may include, but are not limited to, emollients, ointment base, emulsifying agents, solubilizing agents, humectants, thickening or gelling agents, wetting agents, texture enhancers, stabilizers, pH regulators, osmotic pressure modifiers, emulsifiers, UV-A and UV-B screening agents, preservatives, permeation enhancer, chelating agents, antioxidants, acidifying agents, alkalizing agents, buffering agents and vehicle or solvent. In one or more embodiments, the pharmaceutically acceptable carrier includes a liquid, paste or solid form, and more particularly in the form of ointments, creams, milks, pomades, powders, impregnated pads, syndets, solutions, gels, sprays, foams, pastes, suspensions, sticks, shampoos or washing bases. It may also be in the form of suspensions of microspheres or nanospheres or of lipid or polymer vesicles or gelled or polymer patches allowing a controlled release.

The pharmaceutical compositions of any embodiment herein may be formulated for topical administration or any of the routes discussed herein. In one or more embodiments, pharmaceutical compositions may be formulated as a composition for topical administration. The pharmaceutical compositions of the present technology are particularly suited for topical treatment of the skin, and may be in the form of ointments, creams, milks, pomades, powders, impregnated pads, solutions, gels, gel-creams, sprays, lotions, foams or suspensions.

All publications, patents, and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

What is claimed is:

1. A method for treatment of a skin disorder in a subject in need thereof, the method comprising administering said subject, a pharmaceutical composition, wherein said pharmaceutical composition reduces the expression of one or more genes selected from CXCL13, XCL1, SPP1, MMP12 and MMP13.

2. The method according claim 1, wherein the pharmaceutical composition reduces the expression of CXCL13.

3. The method according claim 1, wherein the pharmaceutical composition reduces the expression of XCL1.

4. The method according to claim 1, wherein the pharmaceutical composition reduces the expression of SPP1.

5. The method according claim 1, wherein the pharmaceutical composition reduces the expression of MMP12 and/or MMP13.

6. The method according to claim 1, wherein the pharmaceutical composition comprises a therapeutically effective amount of a pharmaceutical agent and a pharmaceutically acceptable carrier.

7. The method according to claim 1, wherein the pharmaceutical composition comprises from about 1 μg/g to about 100 μg/g of a pharmaceutical agent.

8. The method according to according to claim 6, wherein the pharmaceutical composition is administered once daily, twice daily, once per week, once every two weeks, once every three weeks, once every four weeks, once every five weeks, once every six weeks, once every seven weeks, or once every eight weeks.

9. The method according to claim 1, wherein the pharmaceutical composition is formulated as a cream or as an oil-in-water emulsion.

10. The method according to claim 1, wherein the skin disorder is acne vulgaris.

11. The method according to claim 1, wherein the skin disorder is acne vulgaris of the face or trunk.

12. The method according to claim 10, wherein the acne vulgaris comprises mild, moderate and severe acne vulgaris.

13. The method according to claim 1, wherein the pharmaceutical composition is topically administered to the area of the subject affected with acne vulgaris.

14. A method of modulating an adaptive immune response in a subject to effect a treatment of a skin disorder, comprising administering to the subject a pharmaceutical composition that reduces the expression of one or more genes selected from CXCL13, XCL1, SPP1, MMP12 and MMP13.

15. A method of alleviating inflammatory response associated with skin disorders in a subject, comprising administering to the subject a pharmaceutical composition that reduces the expression of one or more genes selected from CXCL13, XCL1, SPP1, MMP12 and MMP13.

16. The method of claim 15, wherein the skin disorder is acne vulgaris and the method comprises treating acne vulgaris.

17. The method of claim 15, wherein the method decreases inflammation in the skin of the subject.
